(19) United States
(12) Patent Application Publication
(10) Pub. No.: US 2021/0316230 A1
(43) Pub. Date: Oct. 14, 2021

(54) CHLOROPHYLL POLISHING SYSTEMS AND METHODS

(71) Applicant: AerosourceH, LLC, Kevil, KY (US)

(72) Inventor: Charles A. Hall, Metropolis, IL (US)

(73) Assignee: AerosourceH, LLC, Kevil, KY (US)

(21) Appl. No.: 16/846,154

(22) Filed: Apr. 10, 2020

(51) Int. Cl. B01D 15/10; B01D 11/02; B01D 39/18; B01D 35/147; A61K 36/185; C11B 1/04

(52) U.S. Cl. CPC B01D 15/10; B01D 11/0292; B01D 39/18; A61K 2236/31; A61K 36/185; C11B 1/04; B01D 35/1475

(57) ABSTRACT

Infused oil polishing systems and methods reduce or remove chlorophyll from bulk biomass saturated ethanol. A processing vessel includes a valved input port for receiving a biomass mixture and a valved output port for expelling a polished mixture containing the desired extract. The vessel is pressurized or evacuated, causing the chlorophyll-containing carbon to be trapped by the filter assembly as the desired extract mixture is discharged. The equipment operates at relatively low pressures of 40 psi or less, and one operator can run multiple units. The end user can control the degree of polishing by adjusting the amount of media used and resident time. The basic apparatus can also be modified to run in parallel or series combinations to increase throughput. An optional elevating rack provides for ease of cleaning, and no expensive ancillary equipment needs to be purchased.

### FIELD OF THE INVENTION

This invention relates generally to biomass processing and, in particular, to methods and apparatus for removing chlorophyll and other pigments following extraction.

### BACKGROUND OF THE INVENTION

Cannabidiol (CBD) and other Cannabis extracts are now recognized as having valuable medicinal properties. As such, large-scale extraction processes have also grown in importance. The three most popular extraction techniques are alcohol extraction, hydrocarbon extraction, and supercritical CO2 extraction. Alcohol extraction, also commonly referred to as ethanol extraction, is one of the most efficient extraction methods for processing large quantities of biomass.

Regardless of the process used, extraction produces a “crude” product that is subsequently distilled to produce desired extracts. Crude is often unsightly due to the presence of chlorophyll. A deep green uneven coloration is caused by chlorophyll and xanthophyll that are extracted in solution from the biomass. Apart from appearance, chlorophyll is the major cause of downtime in later distillation stages. Accordingly, having an efficient way to remove these substances prior to distillation is advantageous for processors.

The process of removing the chlorophyll is called “polishing.” Existing methods of removing chlorophyll often involve chillers capable of taking the solvents and the mass below −20 C during the extraction process itself. Below such temperatures, the solvent removes less chlorophyll. These chiller systems are expensive to purchase and operate, however, and still leaves appreciable amounts of chlorophyll residues.

The most common cold ethanol extraction process is the quick wash ethanol (QWET) method. Chilling the ethanol has the effect of lessening the solvent's hydrophilic properties, thus minimizing the waxes and pigments that are extracted. However, as temperature is reduced, the level of extracted chlorophyll and plant waxes is reduced. As such, processors must choose between low efficiency or high pigment content.

Activated carbon (or activated charcoal) can also be used to remove chlorophyll and other unwanted non-active pigments in ethanol extracts. However, while existing activated carbon polishing techniques are quite effective at pigment removal, they also remove active compounds. In short, existing activated carbon polishing processes result in a much smaller yield.

There is an outstanding need, therefore, for chlorophyll polishing methods and apparatus that are inexpensive yet highly effective.

### SUMMARY OF THE INVENTION

This invention is directed to infused oil polishing systems and methods designed to reduce or remove chlorophyll from bulk biomass saturated ethanol. The invention improves the quality and usability of the saturate, such that further processing continues with less wear on equipment. The invention is applicable to a wide range of plant-based extracts including cannabinoids.

A polishing system for removing chlorophyll-containing carbon from a biomass mixture according to the invention includes a processing vessel having upper and lower ends. A valved input port at the upper end of the vessel is configured to receive a biomass mixture including a desired extract and chlorophyll-containing carbon particles. A valved output port at the lower end of the vessel is configured for expelling a mixture containing the desired extract. A filter assembly at the lower end of the vessel, in-line and prior to the valved output port, is adapted to receive a replaceable filter material. A port at the upper end of the vessel is adapted for connection to a source of pressurized gas, causing the chlorophyll-containing carbon to be trapped by the filter assembly as the desired extract mixture is discharged through the valved output port.

In preferred embodiments, the processing vessel is an elongated, vertically supported cylindrical vessel that tapers through a lower conical portion to the filter assembly. The filter assembly may be configured to receive one of more pieces of filter paper to retain a charge of silica through which the mixture passes during the polishing process. The system may further include a pressure gauge and/or pressure-relief valve. A plurality of processing vessels may be disposed on a common support structure for series or parallel polishing or other operations.

A method of removing chlorophyll from a biomass mixture includes the step of mixing a predetermined amount of activated carbon with a biomass mixture such that chlorophyll in the biomass mixture attaches to the activated carbon. Replaceable filter materials are loaded into the filter assembly, and the biomass mixture with carbon is introduced into the vessel. The vessel in then pressurized, causing the chlorophyll-attached carbon to be trapped by the filter assembly as the biomass mixture is discharged through the valved output port.

The replaceable filter material may include a filter paper, with the method further including the step of introducing a predetermined quantity of powdered or granularized silica into the process vessel above the filter paper prior to the step of introducing the biomass mixture with chlorophyll-attached carbon into the vessel. The biomass mixture may contain a desired cannabinoid extract.

The apparatus features all food-grade stainless steel construction, as well as the potential for ‘plug-and-play’ expansion. Consumable media and replacement component parts such as gaskets may be obtained from a variety of sources. The equipment operates at relatively low pressures of 40 psi or less, and one operator can run multiple units.

The end user can control the degree of polishing by adjusting the amount of media used and resident time. The basic apparatus can also be modified to run in parallel or series combinations to increase throughput. An optional elevating rack provides for ease of cleaning, and no expensive ancillary equipment needs to be purchased.

### BRIEF DESCRIPTION OF THE DRAWINGS

FIG. 1 is a perspective view of a preferred embodiment of the invention; and

FIG. 2 is a detail view of vessel ports and connectors.

### DETAILED DESCRIPTION OF THE INVENTION

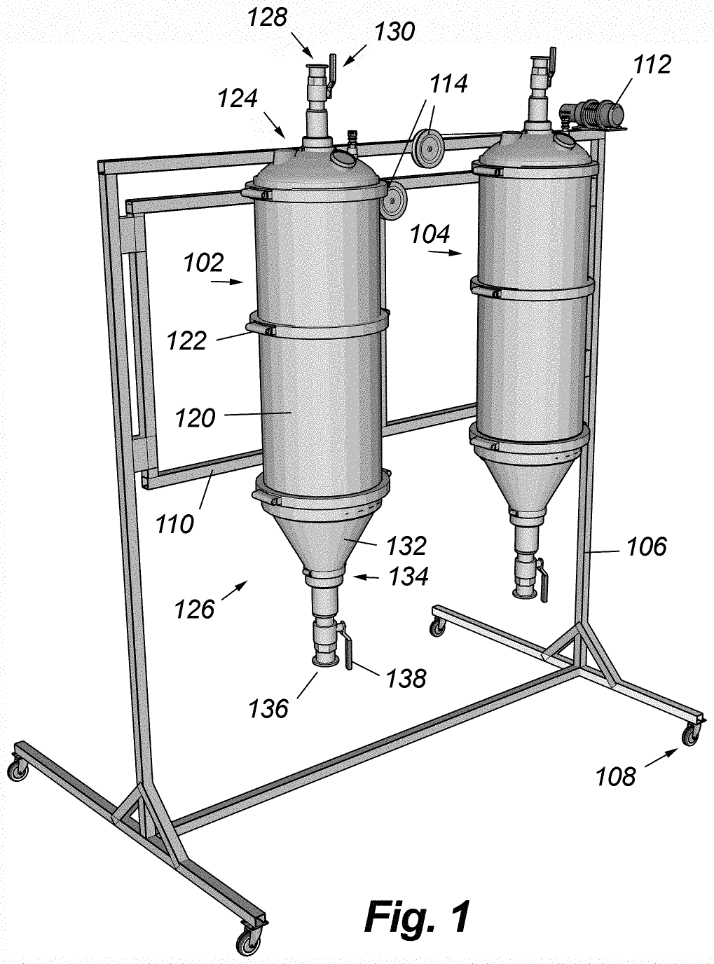

Now making reference to the accompanying drawings, FIG. 1 depicts an embodiment of the invention that incorporates two processing vessels, 102, 104. Actually only one vessel is required; however, two vessels may be provided for enhanced throughput, enabling the two vessels to be used in parallel or in series for the same or different processes described in further detail below.

One or more vessels 102, 104 may be mounted on a frame 106 with casters 108 facilitating ease of use and mobility. The frame 102 may further include a sub frame 110 that translates up and down with winch 112 and casters 114 to move the vessel(s) into different positions for different positions as described below.

Using vessel 102 as an example, the vessel includes a tank 120 that may be constructed with different sections and joined with connectors such as 122. The result, in preferred embodiments, is an elongated vertically oriented cylindrical tank having a diameter of 12 inches, more or less. The completed vessel, including top portion 124, bottom portion 126 and valves, etc., are preferably constructed from food-grade stainless steel.

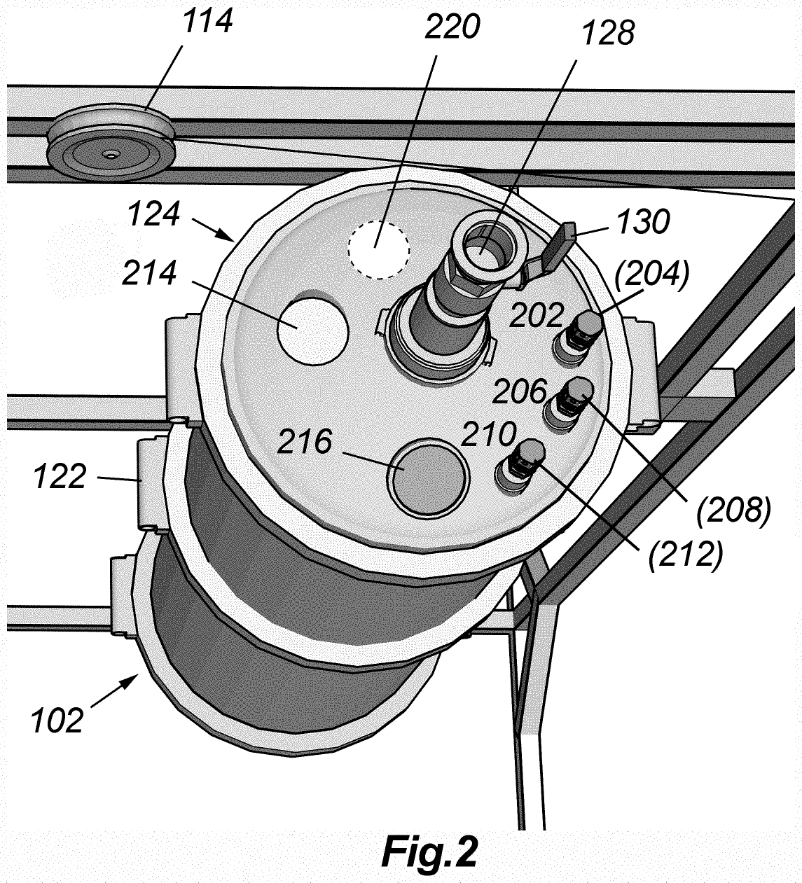

The top portion of 124 includes an inlet pipe 128 with a valve 130. The pipe 128 has a preferred diameter of 1″, and the valve 130 is a quarter-turn shut-off ball valve though other fittings are possible. The top portion 124 includes other components described with reference to FIG. 2. The bottom portion 126 of the vessel 102 preferably includes a conical transition 132 and a filter holder 134. Below the filter holder 134 is a valve 138 that controls discharge through outlet pipe 136. Again, pipe 136 has a preferred diameter of 1″, and the valve 138 is a quarter-turn shut-off ball valve though other fittings are possible here as well. Filter holder 134 is preferably a bell filter holder with a 6″ to 2″ reducer including a 6-inch pressure clamp and gasket.

FIG. 2 is a detail view illustrating the top of a vessel 102. In addition to inlet 128 with valve 130, the top portion includes one or more smaller ports 202, 206, 210. Port 202 connects to a source 204 of pressurized air (or inert gas); port 206 connects to a (60 psi) pressure gauge 208; and port 210 connects to a (60 psi) high-pressure relief valve 212. While three separate ports are shown with individual connects to different items, one or more of the items may be connected to a fewer number of ports into the vessel 102. The top portion of the vessel preferably further includes sight glasses 214, 216. A light is directed into one of the glasses enabling a user to visualize fill level through the other. The top portion of the vessel 102 may also include a capped-off port 220 for future expansion, as discussed below.

### System Operation

Prior to processing, the filter holder 134 is removed and filter paper is installed into holder. For example, #1 and #2 paper (i.e., 11- and 20-micron, respectively) may be used. Other filter papers may be used depending upon desired processing. The filter holder is reassembled and re-installed into the lower discharge port with valve 138 in a closed position. Next, powdered or granulized silica and alcohol are mixed and introduced into the lower funnel portion 126 of the vessel 102 so as to occupy the space immediately above the filter paper. For example, the dry silica may be mixed with 2 gallons of ethanol, stirred well into a slurry, and added to vessel 102 through fill port 128. The slurry need not fill the entire lower conical portion 132 of the vessel 102.

Activated carbon is added to the saturate to be polished prior to introduction into the polishing vessel 102. As one example, about 2 to 2.5 gallons of dry carbon might be added to a full 275-gallon IDC container containing a saturate comprising a mixture of ethanol and biomass (i.e., 90% ethanol and 10% Cannabis extract). The type and porosity of activated carbon is selected in accordance with the saturate, but in most cases, high-quality hardwood charcoal is used with a relatively large pore size, at least for Cannabis processing.

An agitator (not shown) is attached to the IDC container, and the saturate/carbon mixture is agitated for about an hour, then left to sit for about the same length of time. With lower valve 138 closed, the mixture is then introduced into vessel 102 through the upper fill port. Given that the vessel 102 is roughly 20 gallons, several runs may be used to process the entire contents of a full 275-gallon IDC container.

Once filled, the vessel 102 is sealed by closing inlet and outlet valves 130, 138, and the vessel is pressurized. In the preferred embodiment, pressurized air is introduced until the gauge reads 35 psi, though higher and lower pressures may be used as a function of safety versus throughput. At this point the air intake is closed and the lower valve 138 is opened, forcing the saturate through the silica and filter papers, discharging the polished material through the lower port 136. The polished liquid now has the consistency (and appearance) of honey, with the carbon and attached chlorophyll being retained above the silica layer in vessel 102. Note that while it is possible to use suction at the lower end of the apparatus as opposed to pressurization at the upper end, pressurization is cleaner and more efficient. Indeed, it is possible to rely solely on gravity feed, though throughput would be diminished.

The polished material may be dumped into another container or directly into equipment for further processing. For example, the material may be delivered to an evaporator unit to remove ethanol or fed to apparatus of the type just described for further polishing. To clean the vessel 102, a user need only remove the bell filter at the 6″ joint depicted in FIG. 1 at 134. It is possible to re-use or recycle some of the materials following the polishing process. For example, if the filter assembly is removed carefully, the silica and carbon layers may be preserved, enabling the materials to be treated separately. The carbon, for example, may be heated to 700° C., or thereabouts to burn off the chlorophyll and the carbon may be reused.

As mentioned, the apparatus may include a port 220 for future expansion. One such use might be for ‘winterization,’ a process whereby lipids (fats) are removed from crude extract. Lipids are fatty acids extracted from plant material, and winterization is typically the next step after extraction. These unwanted fats dilute desirable fractions, lowering purity, appearance and other desirable qualities. There are various winterization techniques including the use of ethanol, butane and CO2.

‘Cold ethanol’ is perhaps the best extraction method for minimizing fats in extract (though the colder the ethanol, the less fat will be pulled from the plant). At −80° C., for example, most fats and lipids will congeal, and the chlorophyll is transformed into a plastic material, at which point these materials may be more easily removed. The present invention facilitates the use of two vessels; i.e, 102, 104 in FIG. 1, one being used for winterization and the other for polishing, with the output of the winterization vessel being fed directly into the vessel used for polishing.

1. A polishing system for removing chlorophyll-containing carbon from a biomass mixture, the polishing system comprising:
a processing vessel having upper and lower ends;
a valved input port at the upper end of the vessel for receiving a biomass mixture including a desired extract and chlorophyll-containing carbon particles;
a valved output port at the lower end of the vessel for discharging a mixture containing the desired extract;
a filter assembly at the lower end of the vessel for receiving a replaceable filter material, the filter assembly being disposed in-line and prior to the valved output port; and
a port at the upper end of the vessel adapted for connection to a source of pressurized gas causing the chlorophyll-containing carbon to be trapped by the filter assembly as the desired extract mixture is discharged through the valved output port.

2. The polishing system of claim 1, wherein the processing vessel is an elongated, vertically supported cylindrical vessel.

3. The polishing system of claim 2, wherein the vessel tapers through a lower conical portion to the filter assembly.

4. The polishing system of claim 1, wherein filter assembly is configured to receive one of more pieces of filter paper.

5. The polishing system of claim 1, further including a pressure gauge.

6. The polishing system of claim 1, further including a pressure-relief valve.

7. The polishing system of claim 1, further including a plurality of processing vessels disposed on a common support structure.

8. The polishing system of claim 1, wherein the desired extract mixture contains a Cannabis extract.

9. A method of removing chlorophyll from a biomass mixture, comprising the steps of:
mixing a predetermined amount of activated carbon with the biomass mixture such that chlorophyll in the biomass mixture attaches to the activated carbon;
providing the polishing system of claim 1;
loading a replaceable filter material into the filter assembly;
introducing the biomass mixture with carbon into the vessel;
pressurizing the vessel, causing the chlorophyll-attached carbon to be trapped by the filter assembly as the biomass mixture is discharged through the valved output port.

10. The method of claim 9, wherein the replaceable filter material is a filter paper, and wherein the method further includes the step of introducing a predetermined quantity of powdered or granularized silica into the process vessel above the filter paper prior to the step of introducing the biomass mixture with chlorophyll-attached carbon into the vessel.

11. The method of claim 9, wherein the biomass mixture contains a Cannabis extract.
